(19) United States
(12) Patent Application Publication
(10) Pub. No.: US 2020/0397818 A1
(43) Pub. Date: Dec. 24, 2020

(54) Multi-Mineral Supplement for Improved Bone Density

(71) Applicants: AVALON BONE SUPPLEMENT (HK) LIMITED, Shatin (HK); VERSITECH LIMITED, Telegraph Bay (HK)

(72) Inventors: Kelvin Wai Kwok YEUNG, Kennedy Town (HK); Hoi Man WONG, Tsing (HK); Johnson Yiu-Nam LAU, Houston, TX (US); Kenneth Man Chee CHEUNG, Mid Level Central (HK)

(21) Appl. No.: 16/635,481

(22) PCT Filed: Jul. 31, 2017
(86) PCT No.: PCT/US2017/044720

§ 371 Date: Jan. 30, 2020

(51) Int. Cl. A61K 33/34; A61K 33/06; A61K 33/30; A61K 33/00; A61K 9/00; A61K 33/22

(52) U.S. Cl. CPC A61K 33/34; A61K 33/06; A61K 33/30; A61K 9/0053; A61K 9/0024; A61K 33/22; A61K 9/0014; A61K 33/00

(57) ABSTRACT

Compositions and methods are provided in which magnesium, copper, zinc, and strontium are provided to enhance osteogenesis, improve bone growth, and/or improve bone density. Magnesium, copper, zinc, and strontium are provided in a defined molar ratio providing synergistic effects that permit the use of small amounts of individual metal species, and without the need for supplemental calcium.

### FIELD OF THE INVENTION

The field of the invention is mineral supplements useful in improving bone density and/or osteogenesis, particularly mineral supplements containing two or more minerals.

### BACKGROUND

Medical conditions related to bone growth and development, such as osteoporosis, represent a growing contribution to health care costs as the population ages. While some drug therapies have been developed to address such conditions, such pharmaceutical approaches can have drawbacks. For example, hormone replacement therapy is effective in preventing the development of osteoporosis in menopausal women, but can result in an increased risk of the development of certain cancers. More targeted compounds (such as bisphosphonates, which inhibit bone resorption) have been developed, but are associated with hip and thigh fractures. As a result a number of investigators have sought simpler approaches, such as mineral supplementation.

Supplementation with calcium is, by far, the most common therapy applied to loss of bone density. While supplementation with calcium can prove useful success is highly dependent on calcium absorption and utilization by the individual, which in turn may require additional supplementation with vitamins, hormones, and other compounds. As such calcium supplementation alone has limited effectiveness in treatment of conditions related to bone growth and resorption.

Some researchers have investigated treating such disorders with supplementation with various organic compounds, some of which are complexed with metals other than calcium. For example, U.S. Pat. No. 5,294,634, to Yamaguchi, describes the use of carnosine complexed with zinc to promote osteogenesis. All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. U.S. Pat. No. 5,935,996, to Yamaguchi, similarly describes the use of an isoflavone complexed with zinc to promote osteogenesis, and United States Patent Application No. 2008/0113038, to Yamaguchi, describes the use of beta-cryptoxanthin complexed with zinc for this purpose. Similarly, United States Patent Application Publication No. 2010/0048697, to Hansen et al, describes the use of strontium salts of glutamate and alpha-ketoglutarate for treatment of osteoporosis. International Patent Application Publication No. WO 2008/147228, to Cornish and Reid, describes the use of various metal salts of carboxylic acids and carboxylic acid derivatives, along with certain dairy products, in treating conditions related to bone resorption or osteoblast proliferation. Such organic complexes, however, are complex to prepare and can have stability issues.

Attempts have been made to treat conditions related to bone growth using devices or compositions containing metals other than calcium. For example, United States Patent Application Publication No. 2009/0081313, to Aghion et al, describes the use of implants made primarily of elemental magnesium for orthopedic use. In light of the known reactivity of elemental magnesium with water, however, it is not clear if such an approach can provide magnesium in a useable form or in a controlled manner. Similarly, United States Patent Publication No. 2012/0141599, to Johns et al, describes the use of implants that include metal-loaded zeolites for use as bone grafts, specifically citing the use of zinc and copper in combination. In a related approach, United States Patent Application Publication No. 2012/0141429, to Hass, describes the use of zinc oxide coated nanostructures to enhance bone proliferation. While not referring to implants directly, International Patent Application Publication No. WO 2011/058443, to Barralet, describes the application of various insoluble metal phosphate salts at sites where enhanced bone mineralization is desired. It is not clear, however, if such approaches can provide sufficient control of the amount of metal provided in order to avoid the release of toxic and/or cytotoxic amounts.

Thus, there is still a need for compositions and methods for providing a source of metal ions in a controlled fashion to improve bone density.

### SUMMARY OF THE INVENTION

The inventive subject matter provides compositions and methods in which a mixture of Mg, Cu, Sr, and Zn ions is provided to increase bone growth and/or density. While Ca is commonly used as a supplement to support bone growth and development, the Inventors have surprisingly found that Mg, Cu, Sr, and Zn ions, when administered in certain molar ratio, provide a synergistic effect that enhances bone growth and density while permits use of the individual metals in small amounts that are not cytotoxic. Mg, Cu, Sr, and Zn can be provided as an orally administrable form, as a topical formulation, or as an implant.

One embodiment of the inventive concept is a mineral supplement for enhancing bone growth or density. The mineral supplement includes a source of magnesium, a source of copper, a source of zinc, and a source of strontium, and is formulated to provide magnesium, strontium, copper, and zinc to a subject in a Mg:Sr:Cu:Zn molar ratio of 0.8-2.4:5.3-15.8:1:1.3-5. In some embodiments the amounts of these metals present in the supplement can be adjusted by an absorption factor (for example, an oral absorption factor, a topical absorption factor, or an implant absorption factor) to provide the metals at the desired concentrations and/or ratios. The mineral supplement is formulated to provide an increase in bone density or bone volume relative to a corresponding mineral supplement comprising magnesium in the absence of copper, zinc, and strontium. Mg, Sr, Cu, and Zn can be provided as metal salts, metal complexes, and/or metal-containing compounds. In some embodiments the mineral supplement does not include calcium. In other embodiments the source of magnesium is a magnesium salt, a magnesium complex, and/or a magnesium-containing compound. In some embodiments the source of magnesium is not an organic magnesium salt. The source of copper can be a copper salt, a copper complex, and/or a copper-containing compound. In some embodiments the source of copper is not an organic copper salt. The source of zinc can be a zinc salt, a zinc complex, and/or a zinc-containing compound . In some embodiments the source of zinc is not an organic zinc salt. The source of strontium can be a strontium salt, a strontium complex, and/or a strontium -containing compound. In some embodiments the source of strontium is not an organic strontium salt.

In some embodiments such a mineral supplement is formulated as an injectable. In other embodiments the mineral supplement is formulated for oral administration, and the content of individual metals can be adjusted by corresponding oral absorption factors to provide an absorbed Mg:Sr:Cu:Zn molar ratio of 0.8-2.4:5.3-15.8:1:1.3-5. In yet other embodiments the mineral supplement is formulated for topical administration, and the content of individual metals can be adjusted by corresponding topical absorption factors to provide an absorbed Mg:Sr:Cu:Zn molar ratio of 0.8-2.4:5.3-15.8:1:1.3-5. In still other embodiments the mineral supplement is formulated as an implant (which can include an absorbable carrier), and the content of individual metals can be adjusted by corresponding implant absorption factors to provide an absorbed Mg:Sr:Cu:Zn molar ratio of 0.8-2.4:5.3-15.8:1:1.3-5. In some embodiments the implant has a Mg:Cu:Zn:Sr molar ratio of 1.2:7.2-7.9:1:2.5.

Another embodiment of the inventive concept is a method of increasing bone density or improving osteogenesis by providing a mineral supplement comprising a source of magnesium, a source of copper, a source of zinc, and a source of strontium and applying the mineral supplement to an individual in need of increased bone density or improved osteogenesis The mineral supplement is formulated to provide magnesium, copper, zinc, and strontium to the individual at a Mg:Sr:Cu:Zn molar ratio of 0.8-2.4:5.3-15.8:1:1.3-5, and provides an increase in bone density or bone volume relative to a mineral supplement comprising magnesium in the absence of copper, zinc, and strontium. In some embodiments the mineral supplement does not include calcium. In other embodiments the source of magnesium is not an organic magnesium salt, the source of copper is not an organic copper salt, the source of zinc is not an organic zinc salt, and the source of strontium is not an organic strontium salt.

The mineral supplement can be applied by injection or infusion of a solution comprising the mineral supplement. In other embodiments the mineral supplement is applied by topical application of a lotion, gel, suspension, or solution comprising the mineral supplement, and the amounts of specific metals present can be adjusted by corresponding topical absorption factors to achieve the desired Mg:Sr:Cu:Zn ratio. In yet other embodiments the mineral supplement is applied by oral administration of a pill, tablet, capsule, solution, or suspension comprising the mineral supplement, and the amounts of specific metals present can be adjusted by corresponding oral absorption factors to achieve the desired Mg:Sr:Cu:Zn ratio. In still other embodiments the mineral supplement is applied as a surgical implant (where at least part of the surgical implant comprises the mineral supplement), and the amounts of specific metals present can be adjusted by corresponding implant absorption factors to achieve the desired Mg:Sr:Cu:Zn ratio.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIGS. 1A to 1D: FIGS. 1A to 1D depict the results of cell viability studies utilizing human mescenchymal stem cell (hMSCs) cultured with different metal ions at various concentrations. FIG. 1A depicts typical results observed with magnesium ions. FIG. 1B depicts typical results observed with copper ions. FIG. 1C depicts typical results observed with zinc ions. FIG. 1D depicts typical results observed with strontium ions.

FIGS. 2A and 2B: FIGS. 2A and 2B depict normalized alkaline phosphatase (ALP) activity of human mescenchymal stem cell (hMSCs) cultured with different metal ions. FIG. 2A shows typical results for various combinations of Mg, Zn, Cu, and Sr ions at specified concentrations on day 7 and day 14 of exposure. FIG. 2B shows typical results for Mg ion at various concentrations and time points.

FIG. 3: FIG. 3 depicts the position of an implant of the inventive subject matter within a femur of a test subject.

FIGS. 4A and 4B: FIGS. 4A and 4B depict results of in vivo bone growth studies performed using metal ion containing implants. FIG. 4A shows typical Micro-CT images obtained from femurs implanted with the indicated implants at respective time points. FIG. 4B depicts the percentage change in bone volume adjacent to the implants. Significantly more bone is found adjacent to Mg/Sr/Cu/Zn containing implants in weeks 3, 4, and 8 as relative to a Mg-only implant.

FIGS. 5A and 5B: FIGS. 5A and 5B depict results of in vivo bone growth studies performed using metal ion containing implants of the inventive concept having different of Mg, Sr, Cu, and Zn contents. FIG. 5A shows typical Micro-CT images obtained from femurs implanted with the indicated implants at respective time points. FIG. 5B depicts the percentage change in bone volume adjacent to the implants.

FIG. 6A to 6C: FIGS. 6A, 6B, and 6C depict results of in vivo bone growth, femur elastic modulus, and femur stiffness studies performed using metal ion containing implants of the inventive concept having different relative amounts of Mg, Sr, Cu, and Zn ions. FIG. 6A shows typical results for percentage change in cortical bone volume adjacent to the indicated implant compositions at respective time points. FIG. 6B shows typical results for measurements of the elastic modulus of intact femurs implanted with the indicated implant compositions. FIG. 6B shows typical results for measurements of the stiffness of intact femurs implanted with the indicated implant compositions.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Calcium supplementation (generally in the form of orally administered calcium carbonate) has long been used to treat or prevent bone growth disorders, such as osteoporosis in menopause and post-menopause. Surprisingly, Inventors have found that other metals (for example magnesium and other metal salts) can have a significant and unanticipated positive effect on bone growth and density, particularly when administered in combination. While this effect is found in the absence of calcium supplementation such a combination of metals or metal salts can be provided with supplemental calcium and/or other elements (either as part of a mixed metal formulation or administered as an independent formulation).

The inventive subject matter provides compositions and methods in which a mixture of non-calcium metal ions (for example Mg, Cu, Sr, and Zn ions) is provided in amounts and molar ratios that result in increased bone growth and/or density relative to non-treated tissue. While Ca is commonly used as a supplement to support bone growth and development, the Inventors have surprisingly found that Mg, Cu, Sr, and Zn ions, when administered in a specified range of molar ratios, can provide a synergistic (i.e. greater than additive) effect enhances bone growth and density. The synergistic effect so obtained advantageously permits use of the individual metals in non-toxic and/or non-cytotoxic concentrations while providing enhanced bone growth and/or density. Formulations that include Mg, Cu, Sr, and Zn in such molar ratios can be provided with or without supplemental calcium (for example, carbonate, bicarbonate, and/or chloride salts of calcium) and/or other elements (for example, barium, boron, potassium, and/or vanadium). Such supplemental calcium or other elements can form part of an Mg, Cu, Sr, and Zn containing formulation or can be administered separately. Formulations of the inventive concept can be provided as an infusion, an orally administrable form, as a topical formulation, and/or as an implant or artificial graft.

In compositions and methods of the inventive concept a combination of four metallic elements (e.g. magnesium (Mg), strontium (Sr), copper (Cu), and zinc (Zn)) are utilized to induce osteogenesis and/or increase bone density. Such elements can be provided in the form of ionic compounds (i.e. salts), complexes, and/or covalent compounds. In some embodiments these metallic elements are provided using localized delivery (e.g. local application, use of an implant, etc.) rather than systemic delivery (e.g. oral administration, intravenous administration, etc.). While various mineral compositions have been utilized for similar purposes, Inventors have surprisingly found that a synergistic effect on osteogenesis and/or bone density occurs when these metallic elements are provided within a specified range of molar ratios.

One should appreciate that the identification of the synergistic effects of multiple metals in specific ratios in the modification of bone growth advantageously permits the effective use of such metals at individual concentrations well below those associated with cytotoxicity, thereby reducing risk to a person in need of treatment while retaining the therapeutic effects.

While the use of specific salts of magnesium, strontium, copper, and zinc are noted below, it should be appreciated that any suitable inorganic salt of such metals can be used. Examples of suitable anions for such magnesium, strontium, copper, and/or zinc salts include chloride, fluoride, carbonate, bicarbonate, sulfate, phosphate, borate, and/or nitrate. In some embodiments magnesium, strontium, copper, and/or zinc can be provided as oxides. It should be appreciated that the metal salts and/or oxides can be selected to provide the metals at different rates. For example, relatively insoluble magnesium, strontium, copper, and zinc salts (for example, phosphates and/or sulfates) can be selected where a long term effect is desired, such as an implant. Alternatively, relatively soluble magnesium, strontium, copper, and zinc salts (for example, nitrates and/or chlorides) can be selected where an immediate effect is desired, such as an infusion.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Inventors have found that cytotoxic effects can be induced by Mg, Sr, Cu, and/or Zn ions. The MTT colorimetric cell viability provides a straightforward and quantifiable method for determining cell viability in a wide variety of cell types. An MTT assay was used to determine the cytotoxicity of Mg, Sr, Cu, and/or Zn ions in human mescenchymal stem cells. In a typical assay 2.8×10⁴ cells/cm² human mescenchymal stem cell (hMSCs) were cultured in a low glucose DMEM culture medium supplemented with 10% (v/v) fetal bovine serum (FBS, Biowest, France), antibiotics (100 U/ml of penicillin and 100 μg/ml of streptomycin), and 2 mM L-glutamine using 96-well tissue culture plates. After one day of cell culture, testing solutions with a range of concentrations (Table 1) of different potential ions were added to the wells. The MTT solution was prepared by adding thiazolyl blue tetrazolium bromide powder to the phosphate buffered saline (PBS, OXOID Limited, England) and 10 μL of 5 mg/mL MTT solution was added on the next day. 100 μL of 10% sodium dodecyl sulfate (SDS, Sigma, USA) in 0.01 M hydrochloric acid was added to each well after four hours of culturing and incubated at 37° C. in an atmosphere of 5% CO₂ and 95% air overnight. Finally, the absorbance was recorded by a multimode detector at a wavelength of 570 nm with a reference wavelength of 640 nm. Cell viability was quantified from the absorbance readings.

TABLE 1

| Test Species | Test solution | Testing concentration (ppm) |
|---|---|---|
| Magnesium ion (Mg²⁺) | MgCl₂ | 50 to 1,000 |
| Copper ion (Cu²⁺) | CuSO₄ | 5 to 200 |
| Zinc ion (Zn²⁺) | ZnCl₂ | 1 to 400 |
| Strontium ion (Sr²⁺) | SrCl₂ | 50 to 1,000 |

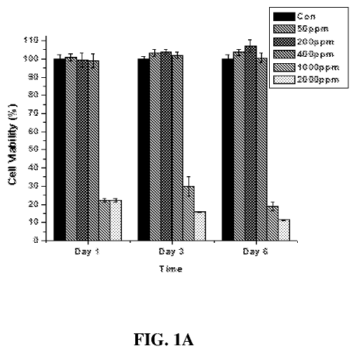

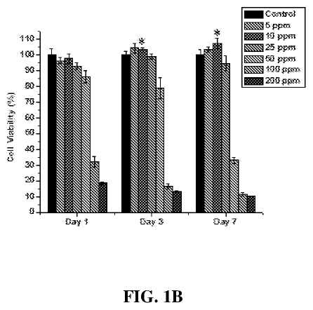

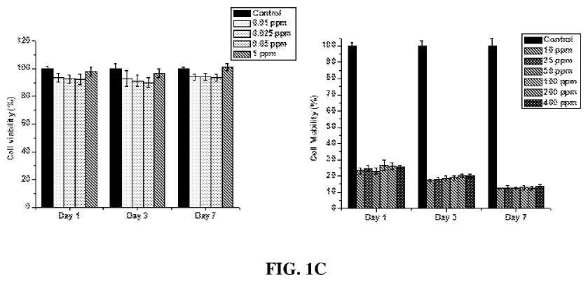

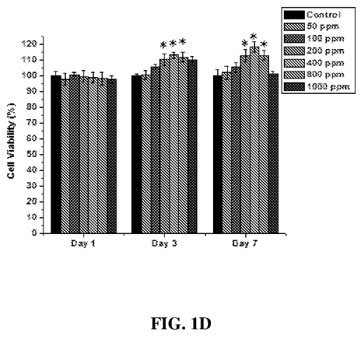

Mg, Sr, Cu, and/or Zn were evaluated for cytotoxicity in the form of ions, and were tested independently to identify effective concentration ranges that did not have cytotoxic effects. Results are shown in FIG. 1A to 1D. FIG. 1A shows typical results of studies performed using magnesium ions. FIG. 1B shows typical results of studies performed using copper ions. FIG. 1C shows typical results of studies performed using zinc ions. FIG. 1D shows typical results of studies performed using strontium ions. Typically, maximum human mescenchymal stem cell (hMSC) viability was observed when the Mg ion concentration was from 50 to 200 ppm and viability began to decrease when the concentration exceeded 400 ppm. Most of the cells became non-viable when the Mg ion concentration exceeded 1000 ppm. Human mescenchymal stem cells were only viable at 5 to 10 ppm Cu ions, with loss of viability found when at a concentration of 25 ppm or higher. Surprisingly, human mescenchymal stem cell (hMSC) were found to be particularly sensitive to Zn ions. Significant cell death was found when Zn ion concentration exceeded 1 ppm. On the other hand human mescenchymal stem cells were found to be tolerant of strontium ions, as treatment with up to 1,000 ppm Sr+ resulted in a cell viability that was close to 100%. Surprisingly, human mescenchymal stem cell viability was typically found increase to 115% (relative to control cells) at Sr ion concentrations of 100 to 400 ppm.

The impact Mg, Sr, Cu, and/or Zn on osteogenic differentiation of human mescenchymal stem cells was determined using alkaline phosphatase (ALP) activity of such cells as an indicator of such differentiation. Accordingly, an ALP assay can be utilized to characterize the osteogenic differentiation properties of Mg, Sr, Cu, and/or Zn, either individually or in combination. In a typical ALP assay 1×10⁴ hMSC cells/cm² were cultured in a low glucose DMEM culture medium supplemented with 10% (v/v) fetal bovine serum (FBS, Biowest, France), antibiotics (100 U/ml of penicillin and 100 μg/ml of streptomycin), and 2 mM L-glutamine on the first day. On the second day, all the culture media in each well was replaced with a differentiation DMEM medium containing 50 μg/ml ascorbic acid (Sigma, USA), 10 mM of β-glycerol phosphate (MP Biomedicals, France), and 0.1 μM dexamethasone (Sigma, USA) together with different combination of Mg, Sr, Cu, and/or Zn ions and incubated at 37 ° C. in an atmosphere of 5% CO₂ for 3, 7 and 14 days. The testing concentration(s) of each ion and testing groups are shown in Table 2 and 3, respectively. These culture media were changed every 3 days. After incubation the cells were washed with phosphate buffered saline (PBS) 3 times and lysed with 0.1% Triton X-100 at 4° C. for 30 minutes. The cell lysates were centrifuged at 574 g and at 4° C. for 10 minutes (2-5 Sartorius, Sigma, USA) and 10 μl of the supernatant of each sample was transferred to a 96-well tissue culture plate. ALP activity was determined by a colorimetric assay using an ALP reagent containing p-nitrophenyl phosphate (p-NPP) (Stanbio, USA) as the substrate. The absorbance was recorded by the multimode detector on the Beckman Coulter DTX 880 at a wavelength of 405 nm. The observed ALP activity was normalized to the total protein concentration of the samples, as determined measured using the Bio-Rad Protein Assay (Bio-Rad, USA).

TABLE 2

| | | Independent | Working |
|---|---|---|---|
| | | effective | concentration (ppm) |
| | Test | concentration | (10% of independent |
| Test Species | solution | (ppm) | working concentration) |
| Magnesium ion (Mg²⁺) | MgCl₂ | 50 | 5 |
| Copper ion (Cu²⁺) | CuSO₄ | 10 | 1 |
| Zinc ion (Zn²⁺) | ZnCl₂ | 1 | 0.1 |
| Strontium ion (Sr²⁺) | SrCl₂ | 400 | 40 |

TABLE 3

| Testing group | Testing concentrations (ppm) |
|---|---|
| Cu only | 10 ppm |
| Sr only | 400 ppm |
| Zn only | 1 ppm |
| Cu + Sr | 1 ppm + 40 ppm |
| Cu + Mg | 1 ppm + 5 ppm |
| Zn + Sr | 0.1 ppm + 40 ppm |
| Zn + Mg | 0.1 ppm + 5 ppm |
| Cu + Sr + Mg | 1 ppm + 40 ppm + 5 ppm |
| Zn + Sr + Mg | 0.1 ppm + 40 ppm + 5 ppm |
| Cu + Zn + Sr + Mg | 1 ppm + 0.1 ppm + 40 ppm + 5 ppm |

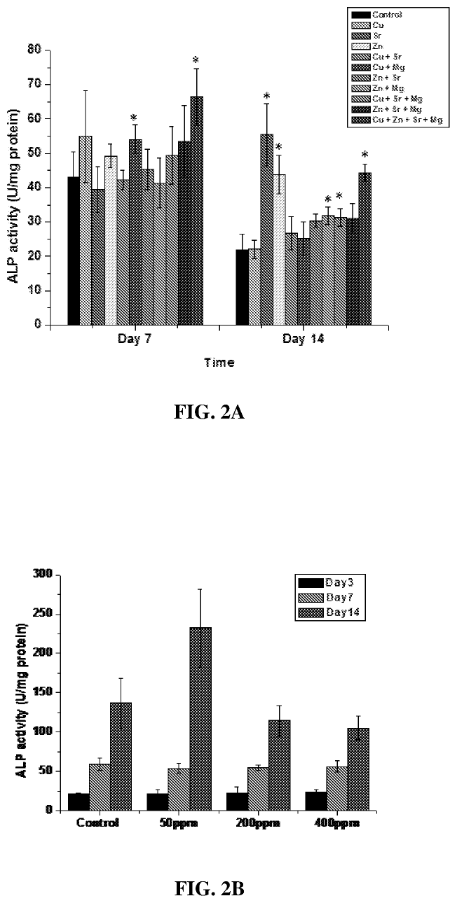

Typical results of osteogenic differentiation studies are shown in FIGS. 2A and 2B. FIG. 2A shows typical normalized ALP activities measured from cells exposed to various combinations and concentrations of Cu, Zn, Sr, and Mg ions. FIG. 2B shows typical results for cells exposed to Mg ions. Surprisingly, treatment with a combination of Cu, Zn, Sr, and Mg ions produced consistent increases in ALP activity relative to control cells (indicative of osteogenic differentiation) for up to 14 days of exposure. This was achieved using Cu, Zn, Sr, and Mg supplied as simple salts, and in the absence of organic salts of these metals (e.g. Cu, Zn, Sr, and/of Mg salts of an organic compound, where the organic compound has a pharmaceutical effect on osteogenesis). The Applicant considers that such differentiation would result in an increase in bone density and/or bone volume in vivo. This consistent increase in osteogenic differentiation was not found for other combinations of two or three of these ions, indicating the combination of Cu, Zn, Sr, and Mg is necessary. In addition the effect was found at concentrations of the individual ions that are far below those found to be cytotoxic. Remarkably, no need for supplemental calcium (Ca) and/or phosphate was found. Similarly, complexation with organic compounds was found to be unnecessary.

Inventors have determined that similar metal element combinations are also effective when delivered locally in vivo. To deliver the combined metal element in vivo, salts of Cu, Zn, Sr, and Mg can be combined with a biocompatible carrier (such as polycaprolactone (PCL). In a typical application specific amounts of selected metal salts can be mixed with PCL. In typical studies implants were fabricated from such materials as rods of 2 mm in diameter by 6 mm in length. The metal content of implants used in initial studies is shown in Table 4.

TABLE 4

| | MgCl₂ | SrCl₂ | CuSO₄ | ZnCl₂ |
|---|---|---|---|---|
| Weight of | 0.01 g | 0.11 g | 0.014 g | 0.03 g |
| Mg/Sr/Cu/Zn | | | | |
| compound in 1 g | | | | |
| PCL carrier | | | | |
| Molar ratio of | 1.20 | 7.91 | 1.00 | 2.51 |
| Mg/Sr/Cu/Zn | | | | |
| PCL carrier | | | | |
| Weight of Mg | 0.1 g | — | — | — |
| compound in 1 g | | | | |
| PCL carrier | | | | |
| (control) | | | | |

In typical studies 2-month old female Sprague-Dawley rats (SD rats) from the Laboratory Animal Unit of The University of Hong Kong were implanted. Their average weights were 200-250 g and the chosen operation site was the lateral epicondyle. Each rat was implanted with the combined ions (Mg/Sr/Cu/Zn)/PCL, or Mg/PCL composites on the right lateral epicondyle. In order to monitor new bone formation around the implants, serial time points of 1, 2, 3, 4 and 8 weeks were set. An Mg/PCL composite served as the control.

The rats were anesthetized with ketamine (67 mg/kg) and xylazine (6 mg/kg) by intraperitoneal injection. The operation sites of the rats were shaved and also underwent decortication. A hole measuring 2 mm in diameter and 6 mm in depth was made by a hand driller at the lateral epicondyle using a minimally invasive approach. Subsequently, the samples were implanted into the prepared holes on the right femur of the rats. The wound was then sutured layer by layer, and a proper dressing was applied over the incision. After the operation, the rats received subcutaneous injections of 1 mg/kg terramycin (antibiotics) and 0.5 mg/kg of ketoprofen. The rats were euthanized in 8 weeks of post-surgery.

At each time point (i.e. 1, 2, 3, 4 and 8 weeks), micro-computed tomography (micro-CT) (SKYSCAN 1076, Skyscan Company) was conducted at the operation site to monitor the healing process and examine new bone formation around the implants. The 2D planes were reconstructed using the NRecon (Skyscan Company).

In some studies, to further evaluate the osteogenic effect of the released ions, the thickness of cortical bone was also measured in addition to the new bone formation around the implant. The limb without implantation was served a control. Micro-CT was conducted at week 4 and week 8 to monitor the new bone formation and cortical bone thickness. The rats were scanned in the micro-CT device (SKYSCAN 1076, Skyscan Company) at these two time points and bone volume was analyzed by the CTAn software (Skyscan Company).

In other studies, flexural stiffness and strength properties of the harvested femurs were determined by using 3-point bending test (MTS 858.02Mini Bionix) based on ASTM D7264-01 standard protocol. The testing speed was 1 mm/min.

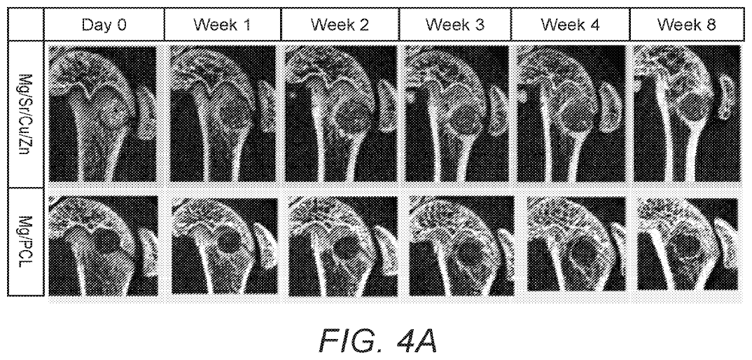

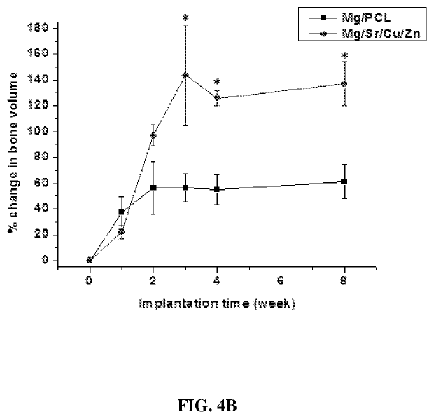

Typical results of initial studies, performed with a single combination of concentrations of Mg, Sr, Cu, and Zn (as described in Table 4) are shown in FIGS. 4A and 4B. FIG. 4A shows typical micro-CT images of rat femurs implanted with the samples from Day 0 to post-op week 8. FIG. 4B shows the percentage change of bone volume adjacent to the sample implants. Typically a 40%-87% increase in bone volume adjacent to the Mg/Sr/Cu/Zn implant (relative to a Mg-only control) was observed in post-op weeks 2, 4, and 8. This indicates that local application of combined Mg, Sr, Cu, and Zn is highly effective in inducing new bone formation. Remarkably, this was achieved without providing supplemental calcium or phosphate. In addition, Cu, Zn, Mg, and Sr were found to be effective when supplied as simple chemical salts (as opposed to metal salts of pharmaceutically active organic compounds).

Similar studies were performed to determine the maximum dosage of metal ions. The sample preparation, surgical procedures and Micro-CT scanning after surgery were essentially identical to those of the studies summarized in FIGS. 4A and 4B. The metal salt content of the implants used are shown in Tables 5A and 5B, which show salt content by weight and ratios of metallic elements to one another, respectively.

TABLE 5A

| | Weight of metallic compounds in 1 g PCL carrier | | | |
|---|---|---|---|---|
| | MgCl₂ | SrCl₂ | CuSO₄ | ZnCl₂ |
| Mg | 0.1 g | N/A | N/A | N/A |
| Mg/Sr/Cu/Zn | 0.01 g | 0.11 g | 0.014 g | 0.03 g |
| (Mg/Sr/Cu/Zn)+ | 0.02 g | 0.2 g | 0.028 g | 0.06 g |
| (Mg/Sr/Cu/Zn)++ | 0.1 g | 1.1 g | 0.14 g | 0.3 g |
| PCL only | N/A | N/A | N/A | N/A |

TABLE 5B

| | Molar ratio of metallic elements PCL carrier | | | |
|---|---|---|---|---|
| | Mg | Sr | Cu | Zn |
| Mg | N/A | N/A | N/A | N/A |
| Mg/Sr/Cu/Zn | 1.20 | 7.91 | 1.00 | 2.51 |
| (Mg/Sr/Cu/Zn)+ | 1.20 | 7.19 | 1.00 | 2.51 |
| (Mg/Sr/Cu/Zn)++ | 1.20 | 7.91 | 1.00 | 2.51 |
| PCL only | N/A | N/A | N/A | N/A |

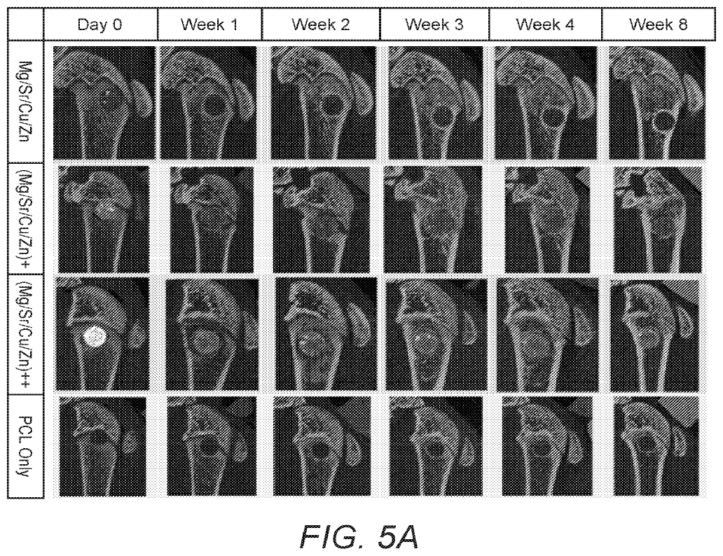

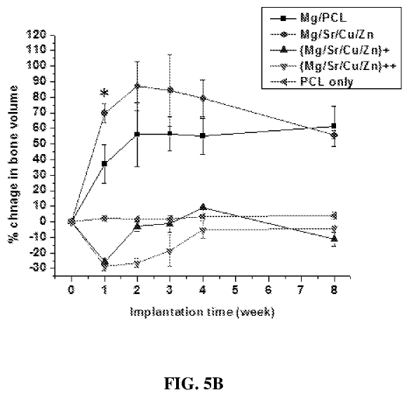

The first and second groups (i.e. Mg and Mg/Sr/Cu/Zn) served to replicate earlier studies, whereas the samples of third (i.e. (Mg/Sr/Cu/Zn)+) and fourth (i.e. (Mg/Sr/Cu/Zn)++) groups were increased with respect to certain metal ions (as shown in Table 5). The increased concentrations ranged from 2-fold to 10-fold higher. The pure PCL group without any metallic ion incorporated served as the control. Typically, at least a 30% to 50% increase of bone volume was found in the Mg/Sr/Cu/Zn group throughout the implantation period as compared with the g Mg/PCL and PCL-only implants, respectively (see FIG. 5). However, 25% or more bone loss was found at one week-post operation when (Mg/Sr/Cu/Zn)+and (Mg/Sr/Cu/Zn)++implants were used. Though the bone loss was reduced at later time points, no new bone formation was identified in association with such implants. This suggests that new bone formation was inhibited at higher concentration metal ion concentrations.

Similar studies were performed to further optimize the concentrations of the metal salt content of the implants. The metal salt compositions of implants utilized in such studies are shown in Table 6A and 6B. Table 6A shows salt content by weight in the implant, whereas Table 6B shows ratios of metallic elements to one another in the implant. In such studies the implants were rods of 1.3 mm in diameter and 2 cm in length. In addition the implant site was the intramedullary of the femur rather than the lateral epicondyle. This permits testing of the mechanical properties of the whole femur.

TABLE 6A

| | Weight of metallic compounds in 1 g PCL carrier | | | |
|---|---|---|---|---|
| | MgCl₂ | SrCl₂ | CuSO₄ | ZnCl₂ |
| Mg/Sr/Cu/Zn | 0.01 g | 0.11 g | 0.014 g | 0.03 g |
| (MSCZ) | | | | |
| (Mg/Sr/Cu/Zn)-1 | 0.01 g | 0.11 g | 0.0105 g | 0.03 g |
| MSCZ-1 | | | (−25%) | |
| (Mg/Sr/Cu/Zn)-2 | 0.01 g | 0.11 g | 0.007 g | 0.003 g |
| MSCZ-2 | | | (−50%) | |
| (Mg/Sr/Cu/Zn)-3 | 0.01 g | 0.11 g | 0.0175 g | 0.003 g |
| MSCZ-3 | | | (+25%) | |
| (Mg/Sr/Cu/Zn)-4 | 0.01 g | 0.11 g | 0.021 g | 0.003 g |
| MSCZ-4 | | | (+50%) | |
| (Mg/Sr/Cu/Zn)-5 | 0.01 g | 0.11 g | 0.014 g | 0.0225 g |
| MSCZ-5 | | | | (−25%) |
| (Mg/Sr/Cu/Zn)-6 | 0.01 g | 0.11 g | 0.014 g | 0.015 g |
| MSCZ-6 | | | | (−50%) |
| (Mg/Sr/Cu/Zn)-7 | 0.01 g | 0.11 g | 0.014 g | 0.0375 g |
| MSCZ-7 | | | | (+25%) |
| (Mg/Sr/Cu/Zn)-8 | 0.01 g | 0.11 g | 0.014 g | 0.045 g |
| MSCZ-8 | | | | (+50%) |

TABLE 6B

| | Molar ratio of metallic elements PCL carrier | | | |
|---|---|---|---|---|
| | Mg | Sr | Cu | Zn |
| Mg/Sr/Cu/Zn | 1.20 | 7.91 | 1.00 | 2.51 |
| (MSCZ) | | | | |
| (Mg/Sr/Cu/Zn)-1 | 1.60 | 10.55 | 1.00 | 3.35 |
| MSCZ-1 | | | | |
| (Mg/Sr/Cu/Zn)-2 | 2.39 | 15.82 | 1.00 | 5.02 |
| MSCZ-2 | | | | |
| (Mg/Sr/Cu/Zn)-3 | 0.96 | 6.33 | 1.00 | 2.01 |
| MSCZ-3 | | | | |
| (Mg/Sr/Cu/Zn)-4 | 0.80 | 5.27 | 1.00 | 1.67 |
| MSCZ-4 | | | | |
| (Mg/Sr/Cu/Zn)-5 | 1.20 | 7.91 | 1.00 | 1.88 |
| MSCZ-5 | | | | |
| (Mg/Sr/Cu/Zn)-6 | 1.20 | 7.91 | 1.00 | 1.25 |
| MSCZ-6 | | | | |
| (Mg/Sr/Cu/Zn)-7 | 1.20 | 7.91 | 1.00 | 3.14 |
| MSCZ-7 | | | | |
| (Mg/Sr/Cu/Zn)-8 | 1.20 | 7.91 | 1.00 | 3.76 |
| MSCZ-8 | | | | |

For the surgical procedures, the distal femur was exposed until the patella is seen. The patella tendon was then displaced to the lateral epicondyle. A hole with 0.5 mm in diameter and 3 mm in length was drilled at the distal end of the right femur through the lateral epicondyle. The sample was then implanted to the drilled hole. The left side (without implantation) was used as the control. The wound was then sutured layer by layer, and a proper dressing was applied over the incision. After the operation, the rats received subcutaneous injections of 1 mg/kg terramycin (antibiotics) and 0.5 mg/kg of ketoprofen. The rats were euthanized 8 weeks of post-surgery.

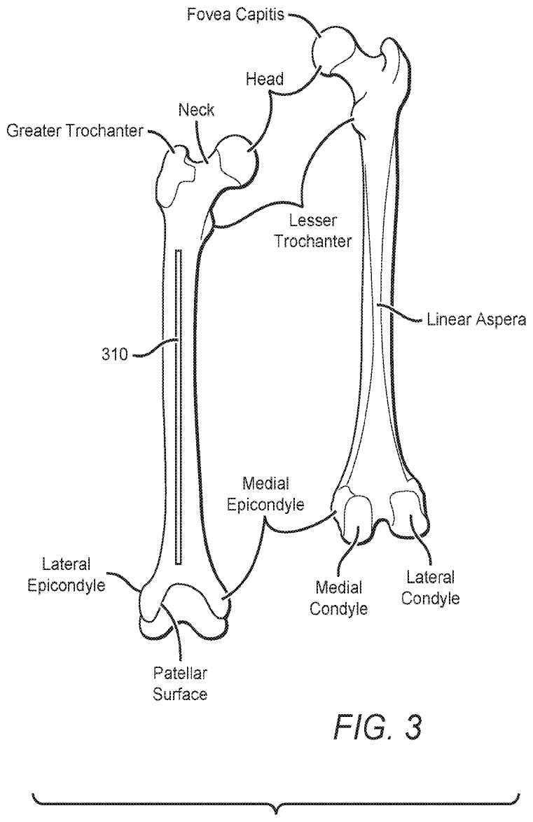

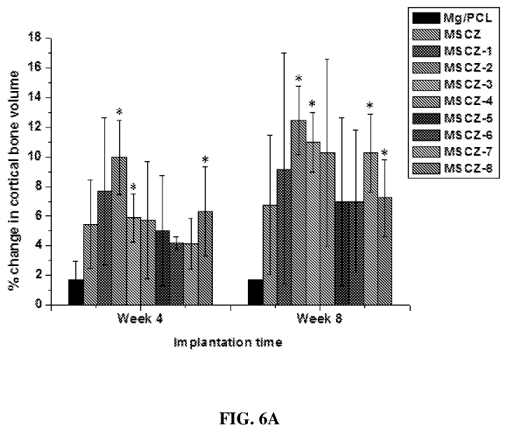

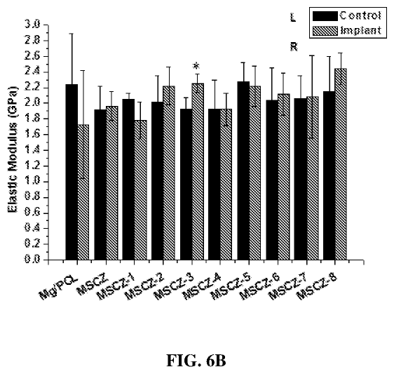

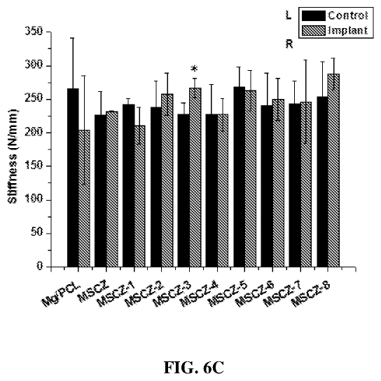

Since magnesium and strontium ions are largely available in the body, the tolerance for these ions can be considered to be relatively high as compared with copper and zinc ions. The inventors, therefore, contemplate that the concentrations of copper and zinc can be critical. Further optimization studies therefore focused on optimization of copper and/or zinc. As noted above, the implants (310) formulated with various amounts of Mg, Sr, Cu, and Zn were surgically implanted into intramedullary canal of femur as shown in FIG. 3 so as to study the effect of the combined metal ions on cortical bone thickness. The non-implanted femur was used as control. Typical results are shown in FIGS. 6A, 6B, and 6C. FIG. 6A shows the percentage of change of the cortical bone volume, and shows that all Mg, Sr, Cu, and Zn formulations tested provided an increase in mean cortical bone volume relative to the non-implanted and Mg-only controls. Surprisingly, the mean bone volume is increased relative to controls for all combinations of Mg, Sr, Cu, and Zn tested. As shown in Table 6B the Mg, Sr, and Zn molar ratios, relative to Cu, can range from 0.8 to 2.4, 5.3 to 15.8, and 1.2 to 5 (respectively) relative to Cu content and provide this effect. In particular, implant formulations MSCZ 2, MSCZ 3, and MSCZ 8 demonstrated a significantly higher cortical bone volume relative to a Mg/PCL control. The increase was approximately by 4-8% in post-op week 4 and 6-11% in post-op week 8. Moreover, implant formulation MSCZ 7 was also able to demonstrate a significantly higher cortical bone thickness, which was approximately 9% higher relative to a Mg/PCL control 8 weeks after implantation. FIG. 6C and FIG. 6C show the elastic modulus and stiffness of the implanted and non-implanted femurs, respectively. The MSCZ 3 implants showed a significantly higher elastic modulus and stiffness when compared with the control. Typically, an increase of 16% in elastic modulus and 14% in stiffness was found in femurs implanted with MSCZ 3 implants relative to the femur without surgical implantation. Therefore, in terms of the cortical bone thickness, mechanical stiffness and elasticity, the MSCZ 2 (Mg:Sr:Cu:Zn=2.4:15.8:1:5), MSCZ 3 (Mg:Sr:Cu:Zn=1.0:6.3:1:2), MSCZ 7 (Mg:Sr:Cu:Zn=1.2:7.9:1:3.1), and MSCZ 8 (Mg:Sr:Cu:Zn =1.2:7.9:1:3.8)implant formulations have particular utility for osteogenesis.

Although embodiments utilizing implants are described above, other embodiments of the inventive concept include methods, devices, and compositions that provide localized delivery of Mg, Sr, Cu, and/or Zn without the use of an implant. In such embodiments Mg, Sr, Cu, and/or Zn can be provided in a flowable formulation, such as a gel or other semisolid, that can be applied at or near a site where increased or enhanced osteogenesis is desired. For example, such a gel or semisolid can be introduced by injection or a minimally invasive surgical procedure. In some embodiments the flowable formulation can solidify following application in order to improve localization. In preferred embodiments such flowable formulations are absorbable.

In other embodiments of the inventive concept, Mg, Sr, Cu, and/or Zn can be provided systemically. Examples of suitable systemic formulations include injectables suitable for parenteral administration, and formulations that can be consumed orally. Suitable orally consumable formulations can be liquid, solid, or semi-solid. In such oral formulations the amounts and/or ratios of Mg, Sr, Cu, and/or Zn provided can be adjusted by an absorption factor in order to provide the desired amount and/or molar ratio of such ions. For example, an absorption factor of about 30% can be applied to water soluble Mg salts that are consumed orally. Similarly, an absorption factor of about 15% to about 30% can be applied to Sr that is consumed orally. An absorption factor of about 20% to 40% can be applied to Zn that is consumed orally. In some embodiments, the absorption factor can vary relative to the amount of Mg, Sr, Cu, and/or Zn provided. For example, the absorption factor for orally ingested Cu can range from about 30% to about 65% as the Cu content decreases. In some embodiments an absorption can vary depending on the specific Mg, Sr, Cu, and/or Zn compound utilized in the formulation. Similar absorption factors can be determined for topical formulations and for implant formulations, and applied to such formulations to provide the desired molar ratio of Cu:Zn:Sr:Mg.

In some embodiments of the inventive concept Mg, Sr, Cu, and/or Zn can be provided along with additional elements that can support bone formation and/or suppress bone resorption. Such additional elements include barium, boron, calcium, potassium, and/or vanadium. Such additional elements can be provided as organic or inorganic salts, complexes, or compounds. For example, barium, calcium, potassium and/or vanadium can be provided as cationic components of salts. Such salts can include an anion such as chloride, fluoride, carbonate, bicarbonate, phosphate, sulfate, borate, or nitrate. In another example, boron can be provided as a borate salt and/or as a boron/carbohydrate complex.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

### REFERENCES

[1] Tao Z S, Zhou W S, He X W, Liu W, Bai BL, Zhou Q, et al. A comparative study of zinc, magnesium, strontium-incorporated hydroxyapatite-coated titanium implants for osseointegration of osteopenic rats. Materials Science & Engineering C-Materials for Biological Applications. 2016; 62:226-32.
[2] He L Y, Zhang X M, Liu B, Tian Y, Ma W H. Effect of magnesium ion on human osteoblast activity. Braz J Med Biol Res. 2016; 49.
[3] Yoshizawa S, Brown A, Barchowsky A, Sfeir C. Magnesium ion stimulation of bone marrow stromal cells enhances osteogenic activity, simulating the effect of magnesium alloy degradation. Acta Biomater. 2014; 10:2834-42.
[4] Castellani C, Lindtner R A, Hausbrandt P, Tschegg E, Stanzl-Tschegg S E, Zanoni G, et al. Bone-implant interface strength and osseointegration: Biodegradable magnesium alloy versus standard titanium control. Acta Biomaterialia. 2011; 7:432-40.
[5] Wang G F, Li J H, Zhang W J , Xu L Y, Pan H Y, Wen J, et al. Magnesium ion implantation on a micro/nanostructured titanium surface promotes its bioactivity and osteogenic differentiation function. International Journal of Nanomedicine. 2014; 9:2387-98.
[6] Meininger S, Mandal S, Kumar A, Groll J, Basu B, Gbureck U. Strength reliability and in vitro degradation of three-dimensional powder printed strontium-substituted magnesium phosphate scaffolds. Acta Biomaterialia. 2016; 31:401-11.
[7] Gentleman E, Fredholm Y C, Jell G, Lotfibakhshaiesh N, O'Donnell M D, Hill R G, et al. The effects of strontium-substituted bioactive glasses on osteoblasts and osteoclasts in vitro. Biomaterials. 2010; 31:3949-56.
[8] Zhang W, Shen Y, Pan H, Lin K, Liu X, Darvell B W, et al. Effects of strontium in modified biomaterials. Acta Biomater. 2011; 7:800-8.
[9] Boda S K, Thrivikraman G, Panigrahy B, Sarma D D, Basu B. Competing Roles of Substrate Composition, Microstructure, and Sustained Strontium Release in Directing Osteogenic Differentiation of hMSCs. ACS Appl Mater Interfaces. 2016.
[10] Yusa K, Yamamoto O, Takano H, Fukuda M, lino M. Zinc-modified titanium surface enhances osteoblast differentiation of dental pulp stem cells in vitro. Sci Rep-Uk. 2016; 6.
[11] Ito A, Otsuka M, Kawamura H, Ikeuchi M, Ohgushi H, Sogo Y, et al. Zinc-containing tricalcium phosphate and related materials for promoting bone formation. Current Applied Physics. 2005; 5:402-6.
[12] Wang T, Zhang J C, Chen Y, Xiao P G, Yang M S. Effect of zinc ion on the osteogenic and adipogenic differentiation of mouse primary bone marrow stromal cells and the adipocytic trans-differentiation of mouse primary osteoblasts. Journal of Trace Elements in Medicine and Biology. 2007; 21:84-91.
[13] Seo H J, Cho YE, Kim T, Shin H I, Kwun I S. Zinc may increase bone formation through stimulating cell proliferation, alkaline phosphatase activity and collagen synthesis in osteoblastic MC3T3-E1 cells. Nutr Res Pract. 2010; 4:356-61.
[14] Jin S J, Ren L, Yang K. Bio-Functional Cu Containing Biomaterials: a New Way to Enhance Bio-Adaption of Biomaterials. Journal of Materials Science & Technology. 2016; 32:835-9.
[15] Wu C T, Zhou Y H, Xu M C, Han P P, Chen L, Chang J, et al. Copper-containing mesoporous bioactive glass scaffolds with multifunctional properties of angiogenesis capacity, osteostimulation and antibacterial activity. Biomaterials. 2013; 34:422-33.
[16] Sen C K, Khanna S, Venojarvi M, Trikha P, Ellison E C, Hunt TK, et al. Copper-induced vascular endothelial growth factor expression and wound healing. Am J Physiol-Heart C. 2002; 282:H1821-H7.
[17] Shi M C, Chen Z T, Farnaghi S, Friis T, Mao X L, Xiao Y, et al. Copper-doped mesoporous silica nanospheres, a promising immunomodulatory agent for inducing osteogenesis. Acta Biomaterialia. 2016; 30:334-44.

What is claimed is:

1. A mineral supplement for enhancing bone growth or density, comprising:
a source of magnesium;
a source of copper;
a source of zinc; and
a source of strontium,
wherein the mineral supplement is formulated to provide magnesium, strontium, copper, and zinc to a subject in a Mg:Sr:Cu:Zn molar ratio of 0.8-2.4:5.3-15.8:1:1.3-5, and
wherein the mineral supplement is formulated to provide an increase in bone density or bone volume relative to a mineral supplement comprising magnesium in the absence of copper, zinc, and strontium.

2. The mineral supplement of claim 1, wherein the mineral supplement does not include calcium.

3. The mineral supplement of claim 1, wherein the source of magnesium is not an organic magnesium salt, the source of copper is not an organic copper salt, the source of zinc is not an organic zinc salt, and the source of strontium is not an organic strontium salt.

4. The mineral supplement of claim 1, wherein the mineral supplement is formulated for application by at least one of injection, oral administration, and topical application.

5. The mineral supplement of claim 4, wherein the content of at least one of the source of magnesium, the source of copper, the source of zinc, and the source of strontium is adjusted by one or more oral absorption factor to provide an absorbed Mg:Sr:Cu:Zn molar ratio of 0.8-2.4:5.3-15.8:1:1.3-5.

6. The mineral supplement of claim 1, wherein the composition is formulated as a surgical implant.

7. The mineral supplement of claim 6, wherein the content of at least one of the source of magnesium, the source of copper, the source of zinc and the source of strontium is adjusted by one or more implant absorption factor to provide an absorbed Mg:Sr:Cu:Zn molar ratio of 0.8-2.4:5.3-15.8:1:1.3-5.

8. The mineral supplement of claim 6, wherein the Mg:Cu:Zn:Sr molar ratio is 1.2:7.2-7.9:1:2.5.

9. The mineral supplement of claim 6, wherein the surgical implant further comprises an absorbable carrier.

10. The mineral supplement of claim 1, further comprising an element selected from the group consisting of barium, boron, calcium, potassium, and vanadium.

11. A method of increasing bone density or improving osteogenesis, comprising:
providing a mineral supplement comprising a source of magnesium, a source of copper, a source of zinc, and a source of strontium; and
applying the mineral supplement to an individual in need of increased bone density or improved osteogenesis,
wherein the mineral supplement is formulated to provide magnesium, copper, zinc, and strontium to the individual at a Mg:Sr:Cu:Zn molar ratio of 0.8-2.4:5.3-15.8:1:1.3-5, and
wherein the mineral supplement is formulated to provide an increase in bone density or bone volume relative to a mineral supplement comprising magnesium in the absence of copper, zinc, and strontium.

12. The method of claim 11, wherein the mineral supplement does not include calcium.

13. The method of claim 11, wherein the source of magnesium is not an organic magnesium salt, the source of copper is not an organic copper salt, the source of zinc is not an organic zinc salt, and the source of strontium is not an organic strontium salt.

14. The method of claim 11, wherein the mineral supplement is applied by injection or infusion of a solution comprising the mineral supplement.

15. The method of claim 11, wherein the mineral supplement is applied by topical application of a lotion, gel, suspension, or solution comprising the mineral supplement.

16. The method of claim 11, wherein the mineral supplement is applied by oral administration of a pill, tablet, capsule, solution, or suspension comprising the mineral supplement.

17. The method of claim 11, wherein the mineral supplement is applied as a surgical implant, wherein at least a portion of the surgical implant comprises the mineral supplement.

18. The method of claim 11, wherein the mineral supplement further comprises an element selected from the group consisting of barium, boron, calcium, potassium, and vanadium.

19. The method of claim 11, further comprising the step of coadministering a supplementary formulation comprising an element selected from the group consisting of barium, boron, calcium, potassium, and vanadium.
